(19) United States
(12) Patent Application Publication
(10) Pub. No.: US 2025/0166828 A1
(43) Pub. Date: May 22, 2025

(54) Medical Prediction Using Loss Functions with Confusion Matrix Terms

(71) Applicant: Picture Health Inc., Cleveland, OH (US)

(72) Inventors: Amogh Hiremath, Pittsburgh, PA (US); Nathaniel Braman, Cleveland, OH (US)

(21) Appl. No.: 18/949,759

(22) Filed: Nov. 15, 2024

Related U.S. Application Data
(60) Provisional application No. 63/600,510, filed on Nov. 17, 2023.

(51) Int. Cl. G16H 50/20; G06T 7/00; G16H 30/40

(52) U.S. Cl. CPC G16H 50/20; G06T 7/0014; G16H 30/40; G06T 2207/30096

(57) ABSTRACT

Machine learning models, such as machine learning classifiers, are trained to make medical predictions using loss functions that include at least one term derived from a confusion matrix, such as negative predictive value (NPV). The medical predictions are based on sets of features extracted from medical images. The medical images may be either radiology images, such as CT, MRI, or PET scans, or pathology images, such as whole-slide images of tissue. The loss functions may include terms that are designed to improve the overall accuracy of the machine learning model in addition to the confusion matrix term or terms. The loss function is typically constructed such that it is continuously differentiable.

### CROSS-REFERENCE TO RELATED APPLICATIONS

This application claims priority to, and the benefit of, U.S. Provisional Patent Application No. 63/600,510, filed Nov. 17, 2023. The contents of that application are incorporated by reference herein in their entirety.

### TECHNICAL FIELD

The invention relates to methods, machine models, and systems for making medical predictions.

### BACKGROUND

Machine vision systems that can make medical predictions from medical images have been investigated in academia and are on the verge of entering clinical medical practice. These systems typically use features extracted from routine clinical radiology or pathology images to make diagnoses, to predict response to particular treatments, to predict the occurrence of complications or side effects, and to predict and project metrics like overall survival. While these systems are applicable to a broad range of medical conditions, they have found initial application in oncology, where the stakes and costs of treatment are high, and accurate medical predictions can thus be of enormous help.

As one example, immune checkpoint inhibitors (ICIs) are a promising class of drugs that allow the body's immune system to attack cancer cells more effectively. However, ICIs have a low overall response rate and a high cost, and administering an ICI to a patient may foreclose the possibility of administering another, potentially more effective, treatment. Thus, accurate predictions of which patients will respond to ICIs and which will not are a great help in clinical practice.

In a typical medical predictive system of this type, a medical image, like a CT image, an MRI image, or a PET scan image, is used to make a medical prediction. The medical prediction can be made either with or without the use of machine deep learning. Using deep learning, the medical image is passed to a deep learning machine model (i.e., a machine model that uses an artificial neural network), or to a collection of such models, and a medical prediction is output. Without deep learning, image features such as radiomic features are extracted from, and sometimes around, a lesion segmented from the medical image. Those features are then sent to a machine-learning classifier to make the medical prediction.

In its most basic form, the output of such a system is usually some risk score indicative of, for example, which patients are likely to respond to ICIs and which patients are not likely to respond. This description uses the term “prediction” because the output of a machine learning model is just that—a prediction. No machine learning model can provide an answer with absolute certainty, and any “yes” or “no” answer that might be provided by such a model reflects the use of thresholds or cut-offs for some underlying risk or probability score.

Usually, machine learning models used for classification are trained and evaluated using the receiver operating characteristic curve (ROC curve). The ROC curve is a plot of the true positive rate versus the false positive rate, and the usual desire is to train and optimize until the area under the ROC curve (the AUC) is as close to 1 as possible. In general, these techniques focus on increasing the accuracy of the whole system, without focusing on any particular outcomes or classes.

### BRIEF SUMMARY

One aspect of the invention relates to a machine learning model implemented on at least one processor and pretrained using a loss function with at least one term derived from a confusion matrix to make one or more medical predictions using radiomic features, pathomic features, or a combination of the radiomic features and the pathomic features. The at least one term derived from the confusion matrix may be a negative predictive value (NPV) term.

Another aspect of the invention relates to a method. The method comprises defining a loss function that includes at least one term derived from a confusion matrix. Using that loss function, the method further comprises training a machine learning model to make one or more medical predictions based on radiomic features, pathomic features, or a combination radiomic features and pathomic features. In the method, the at least one term derived from the confusion matrix may be an NPV term. The at least one of the one or more medical predictions is a negative prediction. The loss function may further include at least one overall model accuracy term, which may be, e.g., a binary cross entropy (BCE) term or a positive predictive value (PPV) term. The terms of the loss function may be weighted, and the loss function may be constructed such that it is continuously differentiable.

Yet another aspect of the invention also relates to a method. The method comprises providing a set of features extracted from medical images to a machine learning model. The machine learning model is pre-trained to make one or more medical predictions using a loss function that has at least one term derived from a confusion matrix. The method also comprises receiving a medical prediction from the machine learning model. The set of features may comprise radiomic features, pathomic features, or a combination of radiomic and pathomic features. The medical prediction may be a negative prediction.

Prior to providing the features to the model, the method may include extracting the set of features from the medical images. Extraction may further comprise constructing a three-dimensional segmentation of one or more of a lesion shown in the medical images, a peri-lesional region, or vasculature associated with the lesion and extract at least some of the set of features from the three-dimensional segmentation.

The loss function may further include at least one overall model accuracy term which may be, e.g., a BCE term or a PPE term. The terms of the loss function may be weighted, and the loss function itself may be continuously differentiable.

In a method according to a further aspect of the invention, medical images may be provided to a deep learning machine model pre-trained to make a medical prediction using a loss function with at least one term derived from a confusion matrix. The method also comprises receiving a medical prediction from the deep learning model.

Yet another further aspect of the invention relates to a system. The system comprises at least one processor, storage coupled to the processor, and a machine learning model. The machine learning model is implemented on the processor and is pre-trained using a loss function with at least one term derived from a confusion matrix to make a medical prediction using one or more medical images.

Other aspects, features, and advantages of the invention will be set forth in the description that follows.

### BRIEF DESCRIPTION OF THE DRAWING FIGURES

The invention will be described with respect to the following drawing figures, in which like numerals represent like features throughout the description, and in which:

FIG. 1 is a schematic illustration of a machine learning system for making a medical prediction based on a medical image or images according to one embodiment of the invention;

FIG. 2 is a set of graphs illustrating the effect of including a weighted negative predictive value (NPV) term in a loss function for a machine learning classifier;

FIG. 3 comprises two sets of box plots, illustrating the effect of an NPV term on the isolation of the negative case;

FIG. 4 is a flow diagram of a method for training a machine learning model to make medical predictions; and

FIG. 5 is a schematic illustration of another machine learning system for making medical predictions.

### DETAILED DESCRIPTION

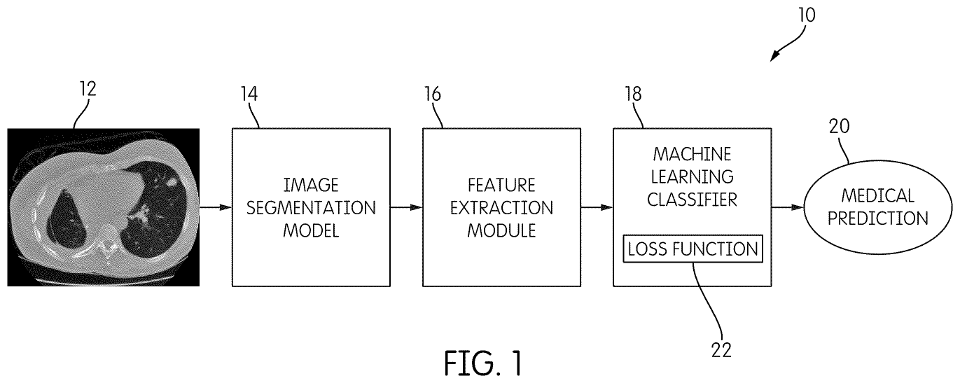

FIG. 1 is a schematic illustration of a machine vision/machine learning system, generally indicated at 10, for making medical predictions based on a medical image or images according one embodiment of the invention. In system 10, a medical image 12 is processed by an image segmentation model 14, features are extracted by a feature extraction module 16, and those features are processed by a machine learning classifier 18 to form some kind of medical prediction 20, which can be reported to a clinician in a variety of ways.

The term “medical prediction” is used here because the raw output of a machine learning model, such as a machine learning classifier 18, is always some sort of probability or risk score, rather than an absolute “yes” or “no” answer. The medical prediction 20 may be, but is not limited to, a diagnosis of a disease or the classification of a disease according to its phenotype or genotype; a prognosis or prediction of disease progression; a prediction of whether a particular lesion is likely to respond to a particular treatment; a prediction of whether the apparent growth of a lesion during treatment represents a true progression of the underlying disease or a pseudo-progression caused by treatment; a prediction of whether a particular patient is likely to experience a particular side effect, like hyper-progression, from a particular treatment; and the like.

In FIG. 1, the medical image 12 is a chest CT image showing one or more lesions. However, the term “medical image,” as used here, refers to any kind of medical imagery, including computed tomography (CT) scans, magnetic resonance imaging (MRI) scans, positron emission tomography (PET) scans, and X-ray images. The term “medical image” also applies to applications of these types of modalities to specific body parts, as in the case of mammography and digital breast tomosynthesis. Typically, in system 10 and other systems and methods according to embodiments of the invention, the medical image 12 will be a routine clinical medical image, i.e., one gathered in the usual course of diagnosis and/or treatment of a disease or condition, rather than an image acquired specifically for use in a system like system 10. However, in some cases, medical images 12 may be gathered specifically for use in system 10.

Additionally, in some embodiments, medical predictions 20 may be made using pathology images, like slide-images of cells or tissues. A hematoxylin and eosin (H&E) stained whole-slide image of a section of a lesion acquired by surgical biopsy, for example, may be used for medical prediction. Thus, pathology images, such as whole-slide images of cells and tissues, should be considered “medical images” for purposes of this description. These sorts of pathology images frequently also fall into the category of routine clinical images, for example, if the tissue was extracted to make or confirm a diagnosis or for some other routine clinical purpose.

Of course, the above assumes that the medical image 12 is a single image. System 10 may produce and use a three-dimensional segmentation from a collection of two-dimensional medical images or from a three-dimensional scan. System 10 may also, in some cases, use multiple medical images acquired over a period of time, e.g., to make a medical prediction using data taken over time. (That is, a so-called “longitudinal study” of one or more patients.) Medical predictions based on, or involving, a patient's progress over time are referred to as longitudinal predictions.

The illustration of FIG. 1 is but one possible embodiment, and the basic tasks described above may be done in different ways or, in some cases, not at all. For example, although a segmentation model 14 and a feature extraction module 16 are shown in FIG. 1, as was noted above, deep learning could also be used. If deep learning is used, a medical image or images 12 could simply be passed to a trained deep learning model, like a convolutional neural network (CNN) or a vision transformer whose output is the medical prediction 20, with no separate segmentation or feature extraction steps or modules. Deep learning models could also be used for some of the tasks, but not others. For example, radiomic or pathomic feature extraction could be carried out and those features input to any traditional machine learning classifier, such as a logistic regression classifier, a support vector machine, etc. Alternatively, a U-net, a type of deep learning model specialized for image segmentation, could be used to perform a segmentation of the medical image 12, and deep features extracted from that model could be sent to a separate classifier 18, which could be either a deep learning classifier 18 or a traditional machine learning classifier based on a statistical technique or operator.

As those of skill in the art will appreciate, not all of the features used to make the medical prediction 20 need be drawn from the medical image 12. For example, patient demographic or history information could also be used, such as the patient's ethnic group or what medications or treatments the patient has previously had. In some cases, a combination of features from different types of medical images, like radiology images and pathology images for the same patient, could be used to make a medical prediction. Other data, like genetic data and histochemical data may also be used.

Overall, the details of medical predictive systems, like system 10, will vary from embodiment to embodiment. The nature of the medical image or images 12 and the nature of the medical prediction 20 are not critical and will vary greatly from embodiment to embodiment. Certain tasks, like separate image segmentation and feature extraction, may be needed in some embodiments and not in others. Yet in all embodiments, there is some type of machine learning model, such as the machine learning classifier 18, that takes some set of features derived from the medical image or images 12 and makes the medical prediction 20. The remainder of this description will focus on how the machine learning classifier 18 is trained and used.

Some degree of error is inherent in the output of any machine learning model. Broadly, in addition to correctly indicating the presence of a condition or attribute (true positive; TP) and correctly identifying the absence of a condition or attribute (true negative; TN), a machine learning classifier may wrongly indicate that a particular condition or attribute is present (false positive; FP) or wrongly indicate that a particular condition or attribute is absent (false negative; FN). These four types of outcomes—TP, TN, FP, and FN—are referred to collectively as the “confusion matrix” for the machine learning classifier and are often represented in a matrix or table form. Most other metrics for evaluating the performance of a machine learning model are derived from the confusion matrix.

As was noted above, a traditional machine learning classifier is trained and evaluated using the receiver operating characteristic curve (ROC curve), which plots the true positive rate (TPR) versus the false positive rate (FPR). TPR, also referred to as sensitivity, is calculated as in Equation (1) below:

$$T P R = \frac{T P}{T P + F N} \qquad ( 1 )$$

FPR is calculated as in Equation (2) below:

$$F P R = \frac{F P}{F P + T N} \qquad ( 2 )$$

As was also noted above, optimizing the ROC curve usually focuses on increasing the accuracy of the system as a whole without targeting specific classes or specific metrics. In some implementations, this is adequate—i.e., all that is needed is a high overall accuracy. In other implementations, a higher-confidence prediction of the positive class or a higher-confidence prediction of the negative class is more desirable.

In clinical medical practice, relying on any prediction from a machine learning system involves some degree of risk; therefore, it may be particularly helpful if the system is able to provide a higher-confidence prediction specifically for the positive class or the negative class. For example, it may be more advantageous and less risky to train the machine learning classifier to provide a high-confidence prediction of which of a group of patients do not have a malignant tumor, rather than training to produce a lower-confidence prediction of which of the group of patients do have a malignant tumor. Similarly, continuing the example of immune checkpoint inhibitors (ICIs) that was given above, it is often better to have a high-confidence prediction that a patient will not benefit from ICIs than a lower-confidence prediction that the patient will benefit from ICIs. That is, it's desirable to avoid a situation in which a machine model predicts that a patient will not respond to ICIs when, in fact, the patient would actually benefit from ICIs. Thus, predicting the group of patients who will not respond to ICIs with high confidence may be more desirable when considering treatment with ICIs. This same risk scenario is found in any number of clinical decisions. Predictions for the negative class are referred to in this description as negative predictions.

In system 10, the machine learning classifier 18 can be adapted and trained using a loss function that includes any metrics derived from the confusion matrix, including those that focus on negative prediction as well as those that focus on positive prediction. Thus, system 10 can be configured to make a much wider variety of types of clinical predictions, and in many cases, will allow a clinical problem to be examined from a different perspective than that of a traditional medical prediction system that is trained to optimize the AUC of the ROC curve for each type of medical prediction. In the present embodiment, this means that the loss function 22 associated with the machine learning classifier 18 has at least one term that takes into account negative outcomes, and the machine learning classifier 18 is thus trained to make negative predictions. (In FIG. 1, the loss function 22 is shown as being a part of the machine learning classifier 18 for case in illustration; as those of skill in the art will understand, a loss function 22 is more properly a tool used to train the machine learning classifier 18. For that reason, portions of this description term the loss function 22 as being “associated with” the machine learning classifier 18.)

Although portions of the remainder of this description may assume that the machine learning classifier 18 is a deep learning classifier, i.e., it uses an a convolutional neural network (CNN), such as a multi-layer perceptron, to make predictions, the techniques described here are equally applicable to machine learning classifiers that use statistical techniques and operators, as these techniques are also generally associated with a loss function (sometimes referred to as a cost function), as well as to other types of machine learning models.

As one example, in the illustrated embodiment, the machine learning classifier 18 has a loss function with a term based on negative predictive value (NPV). In general, NPV is defined as shown in Equation (3) below:

$$N P V = \frac{T N}{F N + T N} \qquad ( 3 )$$

Perhaps the simplest way to construct a loss function, or a term of a loss function, that includes NPV is to binarize the output of the machine learning classifier 18 and then apply Equation (3). However, loss functions are preferably continuously differentiable, such that they can be easily minimized, and the rounding/thresholding operations involved in binarizing the output of the machine learning classifier 18 introduce discontinuities that tend to make the loss function non-differentiable, or at least, not continuously differentiable. Thus, it is preferable to modify the NPV term so that it is continuously differentiable.

For example, if one defines ytrue as a vector of ground truth labels (i.e., binary values of 0 or 1), and ypred is a vector of predictions made by the machine learning classifier 18 in the form of floating-point (i.e., decimal) numbers, a true negative would exist when ytrue=0 and ypred=0, and the sum of true negatives, TN_Sum, can be defined as below in Equation (4):

$$TN\_Sum = \sum ( 1 - y t rue ) ( 1 - ypred ) \qquad ( 4 )$$

Furthermore, with these definitions, a false negative would exist when ytrue=1 and ypred=0, and the sum of false negatives, FN_Sum, can be defined as below in Equation (5):

$$FN\_Sum = \sum ( ytrue ) ( 1 - ypred ) \qquad ( 5 )$$

With TN and FN redefined as above, the definition of NPV, given as Equation (3) above, can be redefined as in Equation (6) below:

$$N P V = \frac{TN\_Sum}{FN\_Sum + TN\_Sum} \qquad ( 6 )$$

The NPV loss term can then be expressed as:

$$NPVloss = 1 - N P V \qquad ( 7 )$$

The above description refers to an NPV term. As those of skill in the art will note, NPV alone does not consider true positives and false positives. In any loss function, there is some term or collection of terms that optimize the performance of the overall model. For example, a binary cross-entropy (BCE) loss function is traditionally used in training deep learning machine classifiers. Because NPV alone does not consider true positives and false positives, alone, it cannot replace a traditional loss function like a BCE loss function. Thus, when it is desirable to tailor a loss function to make a different type of prediction, such as a negative prediction, a term can be added to the typical loss function. For example, if a BCE loss function is used:

$$loss = NPVweight \times NPVloss + BCEweight \times BCEloss \qquad ( 8 )$$

where, in Equation (8), loss is the total loss, NPVloss is as defined above, and BCEloss is the loss calculated with the BCE loss function. There are two other terms: NPVWeight and BCEWeight. These terms are numerical weights applied to the NPV loss and the BCE loss, respectively. The weights give the user the ability to dictate how strongly the negative predictive value factors into the total loss, as compared with the BCE loss.

Although a BCE loss function is typical in some embodiments and implementations, a BCE loss function is not the only type of loss function that may be used with an NPV term, or some other term derived from the confusion matrix. For example, a loss function could be constructed from the weighted sum of the positive predictive value (PPV) and the NPV. The basic definition of PPV is given as Equation (9) below:

$$P P V = \frac{T P}{T P + F P} \qquad ( 9 )$$

However, in practice, PPV would be defined similarly to NPV above, so as to avoid discontinuities that cause problems with continuous differentiability. In general, loss-function terms derived from the confusion matrix may be added to any loss function, with or without weighting.

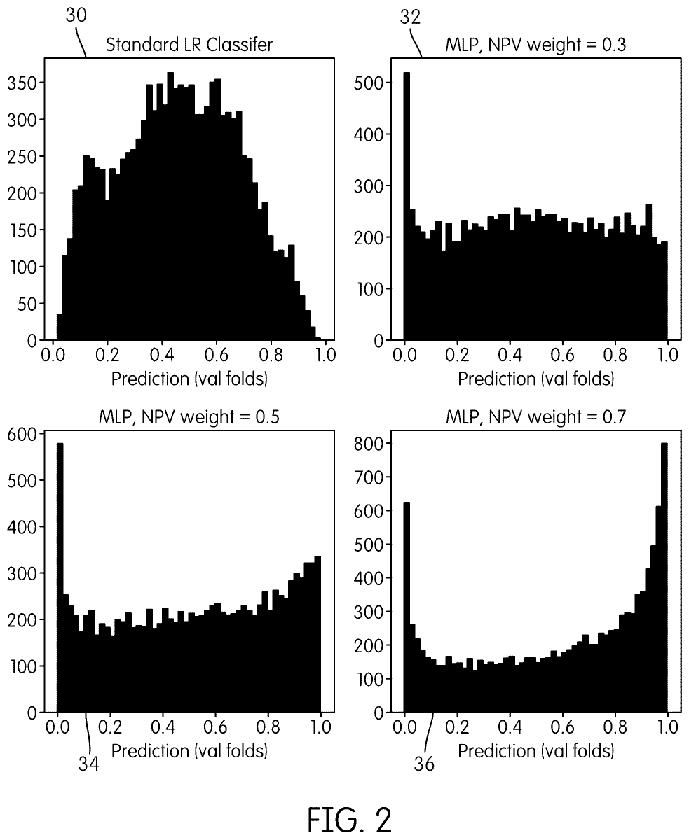

FIG. 2 is a series of graphs illustrating the effect of different weights for the NPV term on the predictions generated by the machine learning classifier 18 in attempting to train the machine learning classifier to distinguish patients who will respond to immunotherapy from those who will not. The graph 30 on the upper right shows the predictions generated by a linear regression classifier with no NPV term in the loss function. These predictions roughly follow a normal distribution, peaking between about 0.3 and 0.7. The graph 32 on the upper right shows the predictions made by a multi-layer perceptron (MLP) using a loss function with an NPV term having a 0.3 weighting. The graph 34 on the lower left shows the predictions made by an MLP using a loss function with an NPV term having a 0.5 weighting. The graph 36 on the lower right shows the predictions made by an MLP using a loss function with an NPV term having a 0.7 weighting. As the NPV term is increased in weight in the loss function, the predictions generated by the MLP classifier increase in confidence, with a much larger number of predictions at and near 0 and 1 and fewer predictions falling in the middle.

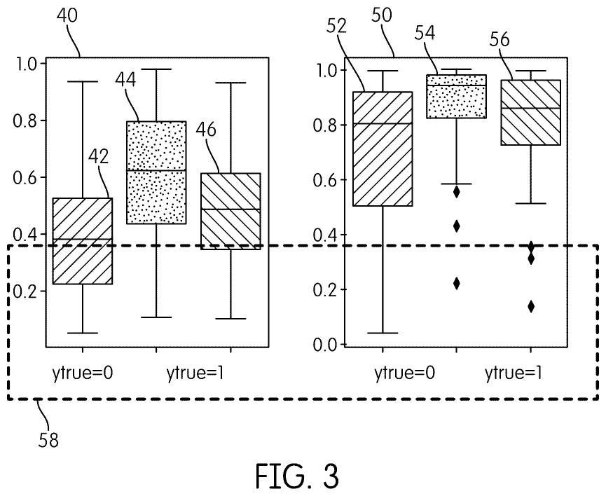

FIG. 3 shows a set of box plots. The set of three box plots 40 on the left is a set of box plots generated using a loss function with no NPV term. Of the three box plots, the negative case box plot 42 is on the left; the other two box plots 44, 46 are of positive cases. The set of three box plots 50 on the right shows predictions made using a loss function with an NPV term. Again, the negative case box plot 52 is on the left; the other two box plots 54, 56 are positive cases.

As shown in these plots, higher NPV is achieved when the positive cases 54, 56 do not have values near zero. Additionally, as the broken-line box 58 encompassing both sets of box plots 40, 50 illustrates, in the box plots 40 generated without an NPV term, there is no region in which, below a certain threshold, only the majority of ytrue=0 exist. In other words, it is not possible to isolate the negative case. However, when an NPV term is used, as in the set of box plots 60, it is possible to isolate the negative case, i.e., to create a threshold below which only the majority of ytrue=0 exist.

Training a machine learning classifier 18 with a loss function that includes a term derived from the confusion matrix, like a PPV term or an NPV term, need not differ substantially from a typical training process. In a typical training process, the machine learning classifier 18 would be trained using a cohort of images with a known outcome relative to the medical prediction 20 that is to be made. For example, if the medical prediction 20 concerns whether a particular lesion seen on a lung CT is benign or malignant, the machine learning classifier 18 would typically be trained using a cohort of medical images with diagnoses confirmed by pathology or other definitive means. If the medical prediction 20 concerns whether a patient with a malignant tumor will respond to ICIs, then the machine learning classifier 18 would typically be trained using a cohort of images for which the patient's response to ICIs is known in each case. As was noted briefly above, the goal in the training process is typically to minimize the value of the loss function. During training, it may be helpful to create several classifier models, each having, e.g., an NPV term with a different weight. Thus, during the training and validation process, the best weight to use can be determined empirically for the data on which the model is to operate.

As those of skill in the art will understand, a training process need not start from nothing. In some cases, techniques like transfer learning can be used, if a model has been trained to make similar predictions. If useful or desirable, multi-task learning can be used to optimize the loss functions of several machine models 14, 18 together.

Once the machine learning classifier 18 has been trained, it is usually validated before being used on medical images 12 for which the outcome is unknown. Validation is usually done by using a second cohort of medical images 12, different than the first cohort, with known outcomes relative to the medical prediction 20 that is to be made. The amount of data (i.e., the number of medical images) necessary for training and validation will depend on the nature of the medical prediction that is to be made, the number of variations expected in the medical images, and other such factors.

The technique of including a term based on the confusion matrix in a loss function for the machine learning classifier 18 has broad application. In particular, the machine learning classifier 18 may be particularly adapted and trained to make medical predictions 20 based on features extracted from the medical image 12. The medical image 12 of FIG. 1 is an image derived from a CT scan, and as such, the features extracted from it are radiomic features. However, radiomic features, pathomic features, or combinations of radiomic and pathomic features may be used.

Typically, radiomic features would be extracted from an area of the medical image 12 encompassing and/or surrounding a lesion identified by the image segmentation model 14. (The region surrounding a lesion is referred to in this text as the “peri-lesional” region.) In this case, the machine learning classifier 18 would essentially be making a medical prediction 20 based on a radiomic examination of the lesion and its surrounding microenvironment.

If a system according to an embodiment of the invention uses a specific feature extraction step, the features that are extracted and used will depend on the nature of the medical prediction 20 that is to be made. In general, the term “extraction” is used to refer to a process of either defining features based on quantitative values in the medical images, or deriving features based on quantitative values in the medical images. The term “features” should be read broadly to include information defined by or derived from quantitative information in medical images, as well as statistics of calculated features. For example, the maximum, minimum, mean, median, skew, kurtosis, etc. of a feature are considered to be features for these purposes.

Examples of radiomic features that may be used include histogram features, textural features, filter- and transform-based features, and size- and shape-based features, including vessel features. Vessel features can be considered to be a special case of size- and shape-based features. The classification of various radiomic features may vary depending on the authority one consults; the categories used here should not be considered a limitation on the range of features that could potentially be used.

Histogram features use the global or local gray-level histogram, and include gray-level mean, maximum, minimum, variance, skewness, kurtosis, etc. Measures of energy and entropy may also be taken as histogram or first-order statistical features. Texture features explore the relationship between voxels, the gray-level cooccurrence matrix (GLCM), the gray-level run-length matrix (GLRLM), gray-level size zone matrix (GLSZM), and gray-level distance zone matrix (GLDZM). Co-occurrence of local anisotropic gradient orientations (COLLAGE) features are another form of texture feature that may be used. (Sec P. Prasanna et al., “Co-occurrence of local anisotropic gradient orientations (collage): distinguishing tumor confounders and molecular subtypes on MRI,” in Int'l Conf. on Med. Image Computing and Computer-Assisted Intervention, pp. 73-80 (Springer, 2014).) Filter- and transform-based features include Gabor features, a form of wavelet transform, and Laws features.

Vessel features, i.e., features of the blood vessels in the peri-lesional region, may be used, including measures and statistics descriptive of vessel curvature and vessel tortuosity. (See, e.g., Braman, N., et al., “Novel Radiomic Measurements of Tumor-Associated Vasculature Morphology on Clinical Imaging as a Biomarker of Treatment Response in Multiple Cancers.” Clin. Cancer Res. 28 (20), pp. 4410-4424, (October 2022).) Transform-based approaches to characterizing vessel features like curvature and tortuosity may also be used, such as Vascular Network Organization via Hough Transform (VaNgOGH). (See, e.g., Braman, N. et al. “Vascular Network Organization via Hough Transform (VaNgOGH): A Novel Radiomic Biomarker for Diagnosis and Treatment Response” in Medical Image Computing and Computer Assisted Intervention—MICCAI 2018 (eds. Frangi, A. F., et al.), pp. 803-811 (Springer, 2018)). As noted above, vessel features can be considered to be a special case of size- and shape-based features, considering the size and shape of the vessels, rather than the lesion. If vessel features are to be used, a segmentation of the vessels around the lesion may be performed. Additional steps may also be taken, like the use of a fast-march algorithm to identify the centerlines of the vessels, and steps to connect disconnected vessel portions.

Pathomic features may include, e.g., features of global and local graphs of the locations of nuclei, nuclear shape features, nuclear orientation entropy, and nuclear texture. Pathomic features may also include measures of other types of cells and structures, including, e.g., graphs and measures of tumor-infiltrating lymphocytes or measures of collagen fiber orientation, as well as statistics and graphs descriptive of these. Pathomic features may also include nuclear shape features, such as nuclear perimeter, minimum and maximum radii, smoothness, and Fourier transform of the nuclear contour (see, e.g., Lu, C. et al. “Nuclear shape and orientation features from H&E images predict survival in early-stage estrogen receptor-positive breast cancers,” Laboratory Investigation 98, pp. 1438-1448 June, 2018); nuclear texture features, such as gray-level co-occurrence features (Ibid.); global graphs of nuclei (see, e.g., Wang, X. et al. “Prediction of recurrence in early stage non-small cell lung cancer using computer extracted nuclear features from digital H&E images,” Scientific Reports 7:13543, October 2017); cell cluster graphs (i.e., local graphs, see, e.g., Ali, S. et al., “Cell cluster graph for prediction of biochemical recurrence in prostate cancer patients from tissue microarrays,” Proc. SPIE 8676, Medical Imaging 2013 March, 2013); cell orientation entropy (CORE; see, e.g., Lee, G. et al., “Cell Orientation Entropy (COrE): Predicting Biochemical Recurrence from Prostate Cancer Tissue Microarrays,” in Medical Image Computing and Computer Assisted Intervention—MICCAI 2013 (eds. Mori, K., et al.), pp. 396-403 (Springer, 2013)); local co-occurrence of cell morphology (LOCOM; see, e.g., Lu, C. et al., “A prognostic model for overall survival of patients with early-stage non-small cell lung cancer: a multicentre, retrospective study,” Lancet Digit. Health 2, e594-606, November 2020); feature-driven local cell clusters (FLocK; see Lu, C. et al., “Feature-driven local cell graph (FLocK): New computational pathology-based descriptors for prognosis of lung cancer and HPV status of oropharyngeal cancers,” Med. Image Analysis 68, November 2020); peri-nuclear pathomics (PNP; see, e.g., Wang, X. et al., “A prognostic and predictive computational pathology image signature for added benefit of adjuvant chemotherapy in early stage non-small-cell lung cancer,” eBioMedicine 69, July 2021); cell run length, which quantifies connectivity and branching patterns of cellular graphs; multinucleation index (MuNI; see, e.g., Koyuncu, C. et al., “Computerized tumor multinucleation index (MuNI) is prognostic in p16+ oropharyngeal carcinoma,” J. Clinical Investigation 131(8), March 2021); spatial interplay of tumor-infiltrating lymphocytes (SpaTIL; see, e.g., Corredor, G. et al., “Spatial Architecture and Arrangement of Tumor-Infiltrating Lymphocytes for Predicting Likelihood of Recurrence in Early Stage Non-Small Cell Lung Cancer,” Clin. Cancer Res. 25(5), March 2019); variations on SpaTIL for gynecologic cancers (ARCTIL; see, e.g., Azarianpour, S. et al., “Computational image features of immune architecture is associated with clinical benefit and survival in gynecological cancers across treatment modalities,” J. Immunother. Cancer 10(2), February 2022); variations on SpaTIL for oropharyngeal cancers (OP-TIL; see, e.g., Corredor, G. et al., “An imaging biomarker of tumor-infiltrating lymphocytes to risk-stratify patients with HPV-associated oropharyngeal cancer,” J. Natl. Cancer Inst. 114(4), pp. 609-617, April 2022); variations on SpaTIL for patients who have received immunotherapy (Histo-TIL; see, e.g., Wang, X. et al., “Spatial interplay patterns of cancer nuclei and tumor-infiltrating lymphocytes (TILs) predict clinical benefit for immune checkpoint inhibitors,” Science Advances 8(22), June 2022); and variations on SpaTIL that quantify TIL sub-populations and the interplay between these populations (PhenoTIL; see, e.g., Barrera, C. et al., “Phenotyping tumor infiltrating lymphocytes (PhenoTIL) on H&E tissue images: predicting recurrence in lung cancer,” Proc. SPIE 10956, Medical Imaging 2019: Digital Pathology 1095607, May 2019).

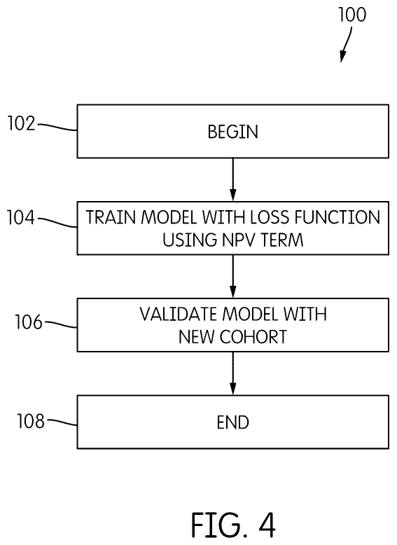

The invention may be embodied in a method, e.g., a method of training a machine model, such as a machine learning classifier, to make a medical prediction based on radiomic, pathomic, or a combination of radiomic and pathomic features. FIG. 4 is a brief, general flow diagram of such a method, generally indicated at 100. Method 100 begins at 102 and continues with task 104. In task 104, the machine model, such as the machine learning classifier 18 described above, is trained. During task 104, a loss function with at least one term derived from the confusion matrix is used in the training, such as a PPV term or an NPV term. In general, one goal of training is to minimize the loss function. As described above, the loss function will typically include other terms as well, and at least the terms may be weighted.

Although much of this description refers to a single model, such as the machine learning classifier 18, several models, e.g., several machine learning classifiers, may be trained at the same time with different loss functions, different cohorts of patient data, etc. At the conclusion of training, the best-performing trained model (e.g., the model with the lowest loss or cost) may be the model selected for validation and functional use. That is, in embodiments of method 100, only the model or model(s) exhibiting the best performance, or, at least, some defined threshold of performance, may continue in method 100.

In task 106, the trained model or models may be validated prior to use by requiring those models to make a medical prediction or medical predictions for a new cohort of patients, not previously seen by the model or models, with known outcomes relative to the medical prediction. If the trained model or models exhibit acceptable results in validation, method 100 ends at 108. If any model does not exhibit acceptable results, it may be retrained with the same or a different loss function, and the same or a different cohort of patient data, in task 104.

FIG. 1 illustrates system 10 in isolation. However, as those of skill in the art will understand, systems like system 10 are usually implemented using computing hardware. In fact, as a general matter, the nature and scale of computation necessary to extract radiomic and pathomic features requires a computer; the computations are too complex to be done in the mind or with pencil and paper, and in many cases, the features themselves are sub-visual.

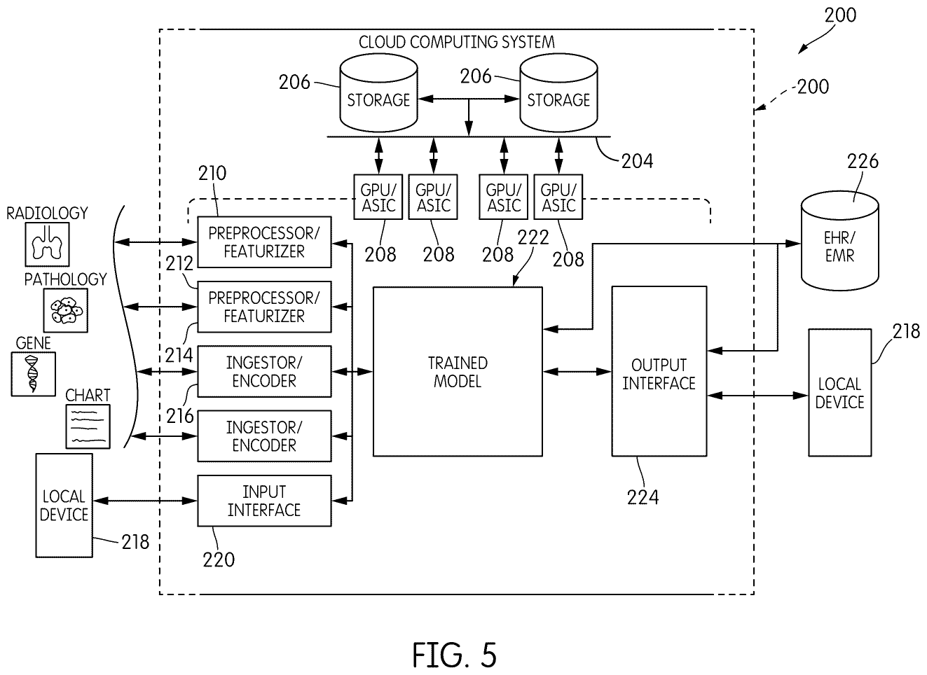

FIG. 5 is an illustration of another system, generally indicated at 200, according to another embodiment of the invention. At the center of system 200 is a cloud computing system 202, i.e., a set of computing hardware that is implemented in a centralized data center and available for dedicated or shared use over a computer network, such as the Internet. The computing hardware typically includes a data bus 204 and a number of storage devices 206 connected to the data bus. The storage devices 206 would typically include mass data storage devices, like hard disk drives, although the computing equipment would also typically include other types of operating memory, such as random access memory (RAM) and read-only memory (ROM), although this is not shown in FIG. 5 for the sake of simplicity.

Also connected to the data bus are a number of processors 208. The processors 208 may be general-use microprocessors, but in many embodiments, the processors 208 will be computer processing elements that are more capable or more specialized for machine learning use, like graphics processing units (GPUs), or application-specific integrated circuits (ASICs), like tensor processing units (TPUs). The remainder of the elements of the cloud computing system 202 are implemented on or by the processors 208.

Among the elements implemented on or by the processors 208 are a number of elements adapted to perform segmentation, to extract radiomic and pathomic features, and to prepare both features and other types of data for use with a machine learning model. These include preprocessor/featurizer elements 210 and 212, which, for purposes of this description, are assumed to be particularly adapted to extract predefined sets of radiomic and pathomic features, as well as ingestor/encoder elements 214, 216, which are adapted to prepare other types of data, like patient demographic data, genetic data, histochemical information, etc. for use by a machine learning model. A local device 218 allows a user to designate the kind of prediction that is desired, the patient or patients for which that prediction is desired, and the information to be used in making the prediction. The local device 218 communicates with an input interface 220.

Prepared information is sent to a machine learning model 222 trained using a loss function with at least one term derived from the confusion matrix, such as a PPV term or an NPV term, to make medical predictions based on the features from the medical images and, optionally, other data. The term “model” is used here, and throughout the description, to refer to a computer program that has been trained to make a prediction or predictions based on various types of input data. Machine learning models may be of various types, and unless the type is specified, the term should be considered to be generic. In this case, the machine learning model may be a machine learning classifier, or it may be a more general deep learning model.

Any output from the model 222 may be passed to an output interface 224. The purpose of the output interface 224 is to take raw output from the model 222 and produce intelligible results, or results that answer a specific prompt or question. An output interface 224 may, for example, apply a threshold or thresholds to raw risk or probability scores to provide an output that a patient is “positive,” “negative” or in one of a number of particular risk categories (e.g., “low risk,” “medium risk,” “high risk,” etc.). In some cases, the output interface 224 may have generative machine learning capabilities. For example, if the medical prediction is whether or not a patient is likely to have relevant gene mutations and should have his or DNA sequenced to confirm those mutations, the output prediction may be a vector comprising 100 binary values indicating whether or not the patient is likely to have each of a number of mutations. The output interface 224 may take that binary information and create a textual or graphical output intelligible to clinicians.

Information from the model 222 may be stored in an electronic health record/electronic medical record (EHR/EMR) 226, either in raw form direct from the model 222, or as processed by the output interface 224. The output interface 224 may also communicate directly with one or more local devices 218. The EHR 226 may be a part of the same cloud computing system 202 as the other components, or it may be a part of a different cloud computing system, i.e., a different software package that communicates with the cloud computing system 202. For example, when a medical prediction is desired from a model 222, it may be ordered in much the same way as a laboratory test using the EHR 226, and the prediction presented in much the same way as a laboratory test.

The invention may also be embodied in a method for making medical predictions. In general, the method may comprise providing a set of features extracted from medical images to a machine learning model pre-trained to make one or more medical predictions using a loss function that has at least one term derived from the confusion matrix; and receiving a medical prediction from the machine learning model.

All of the methods described here may be implemented, at least in part, as software, i.e., a set or sets of machine-readable instructions that, when executed by a machine or machines, like the cloud computing system 202, cause the methods, or portions of them, to be performed.

All references referred to in this text are incorporated by reference in their entireties.

While the invention has been described with respect to certain embodiments, the description is intended to be exemplary, rather than limiting. Modifications and changes may be made within the scope of the invention, which is defined by the appended claims.

What is claimed is:

1. A machine learning model implemented on at least one processor and pretrained using a loss function with at least one term derived from a confusion matrix to make one or more medical predictions using radiomic features, pathomic features, or a combination of the radiomic features and the pathomic features.

2. The machine learning model of claim 1, wherein the at least one term is a negative predictive value (NPV) term.

3. The machine learning model of claim 1, wherein the machine learning model comprises a classifier.

4. The machine learning model of claim 1, wherein the one or more medical predictions comprise one or more of:
a diagnosis of a disease;
a classification of the disease according to phenotype or genotype;
a prognosis or prediction of disease progression;
a prediction of whether a particular lesion is likely to respond to a particular treatment;
a prediction of whether apparent growth of a lesion represents true progression or a pseudo-progression; or
a prediction of whether a particular patient is likely to experience a particular side effect.

5. A method, comprising:
defining a loss function that includes at least one term derived from a confusion matrix; and
using the loss function, training a machine learning model to make one or more medical predictions based on radiomic features, pathomic features, or a combination of radiomic features and pathomic features.

6. The method of claim 5, wherein the at least one term comprises a negative predictive value (NPV) term.

7. The method of claim 6, wherein at least one of the one or more medical predictions is a negative prediction.

8. The method of claim 5, wherein the loss function further includes at least one overall model accuracy term.

9. The method of claim 8, wherein the overall model accuracy term comprises a binary cross entropy (BCE) term or a positive predictive value (PPV) term.

10. The method of claim 8, wherein the at least one at least one term and the overall model accuracy term are weighted.

11. The method of claim 5, wherein the loss function is continuously differentiable.

12. A method, comprising:
providing a set of features extracted from medical images to a machine learning model pre-trained to make one or more medical predictions using a loss function that has at least one term derived from a confusion matrix; and
receiving a medical prediction from the machine learning model.

13. The method of claim 12, wherein the set of features comprises radiomic features, pathomic features, or a combination of radiomic and pathomic features.

14. The method of claim 12, further comprising:
prior to said providing, extracting the set of features from the medical images.

15. The method of claim 14, wherein said extracting comprises:
constructing a three-dimensional segmentation of one or more of: a lesion shown in the medical images, a peri-lesional region, or vasculature associated with the lesion; and
extracting at least some of the set of features from the three-dimensional segmentation.

16. The method of claim 12, wherein the loss function further includes at least one overall model accuracy term.

17. The method of claim 16, wherein the overall model accuracy term comprises a binary cross entropy (BCE) term or a positive predictive value (PPV) term.

18. The method of claim 17, wherein the at least one term and the overall model accuracy term are weighted.

19. The method of claim 12, wherein the loss function is continuously differentiable.

20. The method of claim 12, wherein the at least one term comprises a negative predictive value (NPV) term.

21. The method of claim 20, wherein at least one of the one or more medical predictions is a negative prediction.

22. A set of machine-readable instructions on a machine-readable medium that, when executed, cause the machine to perform the method of claim 12.

23. A method, comprising:
providing one or more medical images to a deep learning machine model pre-trained using a loss function with at least one term derived from a confusion matrix to make a medical prediction based on the one or more medical images; and
receiving a medical prediction from the deep learning machine model.

24. A system, comprising:
at least one processor;
storage coupled to the processor; and
a machine learning model implemented on the at least one processor and pre-trained using a loss function with at least one term derived from a confusion matrix to make a medical prediction using one or more medical images.

25. The system of claim 24, wherein the machine learning model is pre-trained to make the medical prediction using features extracted from the one or more medical images.

26. The system of claim 25, wherein the features comprise radiomic features, pathomic features, or a combination of radiomic and pathomic features.

27. The system of claim 26, further comprising:
at least one feature extraction module adapted to extract the features from the medical images.

28. The system of claim 24, wherein the at least one term comprises a negative predictive value term.

29. The system of claim 28, wherein the medical prediction is a negative prediction.
